(19) United States
(12) Patent Application Publication
(10) Pub. No.: US 2018/0338745 A1
(43) Pub. Date: Nov. 29, 2018

(54) ULTRASOUND DIAGNOSIS APPARATUS AND ULTRASOUND DIAGNOSIS AIDING APPARATUS

(71) Applicant: Canon Medical Systems Corporation, Otawara-shi (JP)

(72) Inventor: Takashi TAKEUCHI, Otawara (JP)

(73) Assignee: Canon Medical Systems Corporation, Otawara-shi (JP)

(21) Appl. No.: 15/991,346

(22) Filed: May 29, 2018

(30) Foreign Application Priority Data
May 29, 2017 (JP) 2017-105577

(51) Int. Cl. A61B 8/00; A61B 34/32; A61B 90/00; A61B 8/08

(52) U.S. Cl. CPC A61B 8/4218; A61B 34/32; A61B 90/06; A61B 8/543; A61B 2090/064; A61B 8/5223; A61B 8/4263; A61B 8/465; A61B 8/463; A61B 8/488

(57) ABSTRACT

An ultrasound diagnosis apparatus according to an embodiment includes an ultrasound probe, a robot arm, and processing circuitry. The ultrasound probe is configured to transmit and receive an ultrasound wave. The robot arm is configured to hold the ultrasound probe and to move the ultrasound probe along the body surface of a subject. The processing circuitry is configured to control the moving of the ultrasound probe performed by the robot arm. The processing circuitry is configured to exercise control so that an instruction for the subject is output on the basis of instruction information related to an ultrasound diagnosis process.

### CROSS-REFERENCE TO RELATED APPLICATIONS

This application is based upon and claims the benefit of priority from Japanese Patent Application No. 2017-105577, filed on May 29, 2017; the entire contents of which are incorporated herein by reference.

### FIELD

Embodiments described herein relate generally to an ultrasound diagnosis apparatus and an ultrasound diagnosis aiding apparatus.

### BACKGROUND

Conventionally, ultrasound diagnosis processes are performed by obtaining information about a tissue structure, a blood flow, or the like on the inside of a human body, as a technologist or a medical doctor operates an ultrasound probe on the body surface of the subject. For example, in accordance with the diagnosed site or the content of a diagnose, the technologist or the medical doctor scans the inside of the body of the subject with an ultrasound wave by operating, on the body surface, the ultrasound probe configured to transmit and receive the ultrasound wave, so as to acquire an ultrasound image exhibiting the tissue structure or an ultrasound image exhibiting the information about the blood flow or the like.

To perform such ultrasound diagnosis processes, having a scan performed by a robot has been proposed in recent years. For example, an ultrasound probe is held by a robot arm and is operated on the body surface of a subject, so as to acquire an ultrasound image exhibiting a tissue structure or an ultrasound image exhibiting information about a blood flow or the like.

### BRIEF DESCRIPTION OF THE DRAWINGS

FIG. 1 is an external view of an ultrasound diagnosis apparatus according to a first embodiment;

FIG. 2 is a block diagram illustrating an exemplary configuration of the ultrasound diagnosis apparatus according to the first embodiment;

FIG. 3 is a table illustrating an example of correspondence information according to the first embodiment;

FIG. 4A is a drawing illustrating an example of output information output by an output controlling function according to the first embodiment;

FIG. 4B is a drawing illustrating another example of the output information output by the output controlling function according to the first embodiment;

FIG. 5 is a flowchart for explaining a procedure in a process performed by the ultrasound diagnosis apparatus according to the first embodiment;

FIG. 6 is a block diagram illustrating an exemplary configuration of an ultrasound diagnosis apparatus according to a second embodiment;

FIG. 7 is a flowchart for explaining a procedure in a process performed by the ultrasound diagnosis apparatus according to the second embodiment;

FIG. 8 is a block diagram illustrating an exemplary configuration of an ultrasound diagnosis apparatus according to a third embodiment; and

FIG. 9 is a block diagram illustrating an exemplary configuration of an ultrasound diagnosis aiding apparatus according to a fourth embodiment.

### DETAILED DESCRIPTION

According to an embodiment, an ultrasound diagnosis apparatus includes an ultrasound probe, a robot arm and processing circuitry. The ultrasound probe is configured to transmit and receive an ultrasound wave. The robot arm is configured to hold the ultrasound probe and to move the ultrasound probe along a body surface of a subject. The processing circuitry is configured to control the moving of the ultrasound probe performed by the robot arm. The processing circuitry is configured to exercise control so that an instruction for the subject is output on a basis of instruction information related to an ultrasound diagnosis.

Exemplary embodiments of an ultrasound diagnosis apparatus and an ultrasound diagnosis aiding apparatus of the present disclosure will be explained in detail below, with reference to the accompanying drawings. Possible embodiments of the ultrasound diagnosis apparatus and the ultrasound diagnosis aiding apparatus of the present disclosure are not limited by the embodiments described below. Further, in the following explanations, some of the constituent elements that are the same as each other will be referred to by using the same reference characters, and the duplicated explanations thereof will be omitted.

### First Embodiment

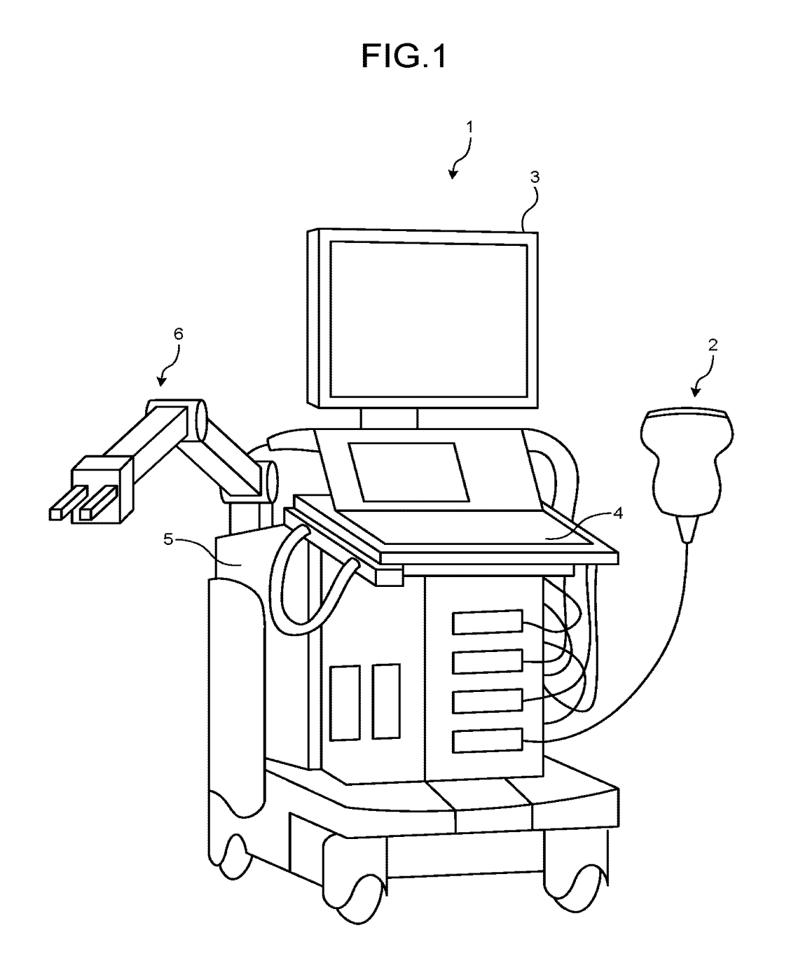

To begin with, an ultrasound diagnosis apparatus according to a first embodiment will be explained. FIG. 1 is an external view of an ultrasound diagnosis apparatus 1 according to the first embodiment. As illustrated in FIG. 1, the ultrasound diagnosis apparatus 1 according to the first embodiment includes an ultrasound probe 2, a monitor 3, an input interface 4, an apparatus main body 5, and a robot arm 6.

The ultrasound probe 2 has a probe main body and a cable and is connected to the apparatus main body 5 via the cable. Further, on the basis of a drive signal supplied thereto from a transmission and reception circuit (explained later), the ultrasound probe 2 is configured to cause an ultrasound wave to be generated from a plurality of piezoelectric transducer elements included in the probe main body, to transmit the generated ultrasound wave to the inside of an examined subject, and to receive a reflected wave occurring as a result of the transmitted ultrasound wave being reflected on the inside of the subject. In this situation, the ultrasound probe 2 according to the first embodiment is configured so that the probe main body is held by the robot arm 6 and is moved along the body surface of the subject.

The monitor 3 is configured to display a Graphical User Interface (GUI) used by an operator of the ultrasound diagnosis apparatus 1 to input various types of setting requests through the input interface 4 and to display various types of images generated by the apparatus main body 5. Further, the monitor 3 is configured to output instruction information for the subject on the basis of control exercised by the apparatus main body 5. For example, the monitor 3 displays the instruction information realized with text, animation, or the like for the subject. In another example, the monitor 3 outputs the instruction information realized with audio from a speaker built therein. The instruction information provided for the subject will be explained in detail later.

The input interface 4 is realized by using a mouse, a keyboard, a button, a panel switch, a touch command screen, a trackball, a joystick, a microphone, and/or the like. The input interface 4 is configured to receive the various types of setting requests from the operator of the ultrasound diagnosis apparatus 1 and to transfer the received various types of setting requests to the apparatus main body 5. Further, the input interface 4 is configured to receive a request from the subject and to transfer the received request to the apparatus main body 5. The request made by the subject will be explained in detail later.

The apparatus main body 5 is configured to control the entirety of the ultrasound diagnosis apparatus 1. For example, the apparatus main body 5 is configured to generate an ultrasound image on the basis of the reflected-wave signal received by the ultrasound probe 2. Further, the apparatus main body 5 is configured to perform various types of processes in response to the request received by the input interface 4.

The robot arm 6 includes a holding unit (a probe holder) configured to hold the probe main body of the ultrasound probe 2 and a mechanism unit for moving the ultrasound probe 2 (the probe main body) to a desired position on the body surface of the subject. In other words, the robot arm 6 is configured to move the ultrasound probe 2 held by the holding unit to the desired position with a movement of the mechanism unit. For example, as illustrated in FIG. 1, the robot arm 6 is attached to the top face of the apparatus main body 5 so as to move the ultrasound probe 2 in accordance with control exercised by the apparatus main body 5. In this situation, the apparatus main body 5 makes it possible for the ultrasound probe 2 to move along the body surface of the subject, by moving the robot arm 6 on the basis of a computer program (hereinafter, “program”) set in advance.

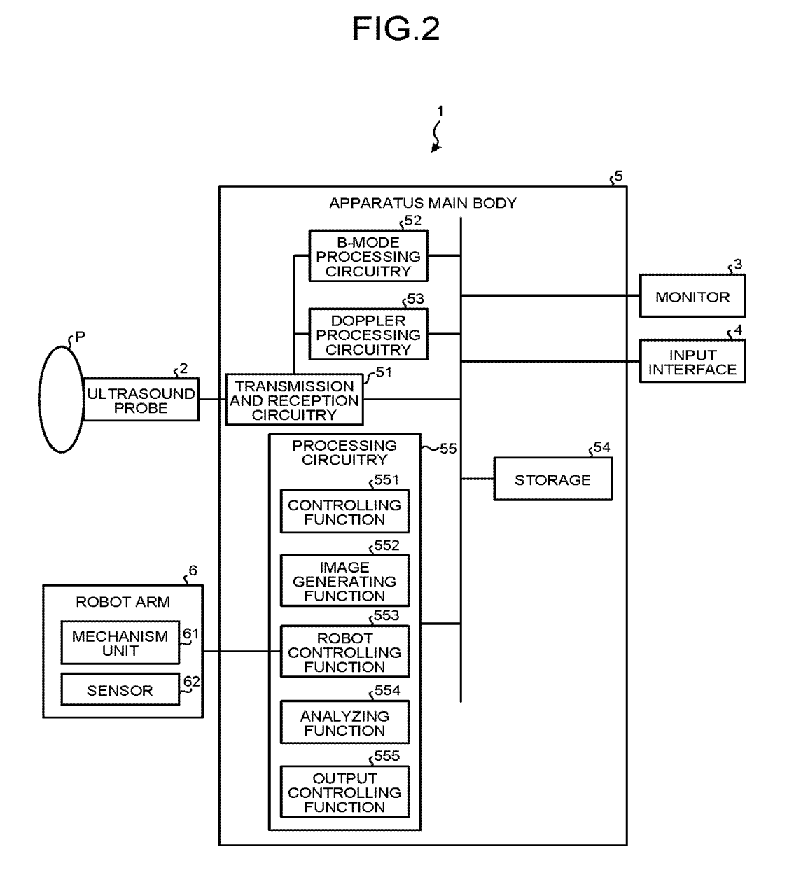

As explained above, the ultrasound diagnosis apparatus 1 according to the first embodiment is configured to generate the ultrasound image by scanning a target site of the subject, by arranging the ultrasound probe 2 to be held by the robot arm 6 and moving the robot arm 6 in accordance with the program set in advance. Next, a detailed configuration of the ultrasound diagnosis apparatus 1 will be explained. FIG. 2 is a block diagram illustrating an exemplary configuration of the ultrasound diagnosis apparatus 1 according to the first embodiment. As illustrated in FIG. 2, the ultrasound diagnosis apparatus 1 according to the present embodiment is configured so that the ultrasound probe 2, the monitor 3, the input interface 4, and the robot arm 6 are connected to the apparatus main body 5.

The ultrasound probe 2 is connected to transmission and reception circuitry 51 included in the apparatus main body 5. For example, the ultrasound probe 2 includes the plurality of piezoelectric transducer elements provided in the probe main body. Each of the plurality of piezoelectric transducer elements is configured to generate an ultrasound wave on the basis of the drive signal supplied thereto from the transmission and reception circuitry 51. Further, the ultrasound probe 2 is configured to receive a reflected wave from a subject P and to convert the received reflected wave into an electrical signal. Further, the ultrasound probe 2 includes, within the probe main body, matching layers provided for the piezoelectric transducer elements, as well as a backing member or the like that prevents the ultrasound waves from propagating rearward from the piezoelectric transducer elements. In this situation, the ultrasound probe 2 is detachably connected to the apparatus main body 5. For example, the ultrasound probe 2 is an ultrasound probe of a sector type, a linear type, or a convex type.

When an ultrasound wave is transmitted from the ultrasound probe 2 to the subject P, the transmitted ultrasound wave is repeatedly reflected on a surface of discontinuity of acoustic impedances at a tissue in the body of the subject P and is received as a reflected-wave signal by each of the plurality of piezoelectric transducer elements included in the ultrasound probe 2. The amplitude of the received reflected-wave signal is dependent on the difference between the acoustic impedances on the surface of discontinuity on which the ultrasound wave is reflected. When a transmitted ultrasound pulse is reflected on the surface of a moving blood flow, a cardiac wall, or the like, the reflected-wave signal is, due to the Doppler Effect, subject to a frequency shift, depending on a velocity component of the moving members with respect to the ultrasound wave transmission direction.

The present embodiment is applicable to both the situation in which the subject P is two-dimensionally scanned by the ultrasound probe 2 realized with a one-dimensional ultrasound probe in which the plurality of piezoelectric transducer elements are arranged in a row; and the situation in which the subject P is three-dimensionally scanned by the ultrasound probe 2 where the plurality of piezoelectric transducer elements included in a one-dimensional ultrasound probe are mechanically caused to swing or by the ultrasound probe 2 realized with a two-dimensional ultrasound probe in which the plurality of piezoelectric transducer elements are two-dimensionally arranged in a grid formation.

As illustrated in FIG. 2, the robot arm 6 includes a mechanism unit 61 and a sensor 62. For example, as illustrated in FIG. 1, the mechanism unit 61 includes a plurality of arm units and a plurality of joints, while the arm units are linked to one another by the joints. In this situation, for example, the mechanism unit 61 is configured so that the joints are provided with actuators. As a result of the actuators operating under the control of the apparatus main body 5, the mechanism unit 61 moves the ultrasound probe 2 to a desired position on the body surface of the subject. In this situation, the number of joints as well as the types and the number of actuators provided for the joints may arbitrarily be determined. In other words, the degree of freedom of the joints of the robot arm 6 may arbitrarily be set (e.g., to have six or more axes). In one example, as illustrated in FIG. 1, the mechanism unit 61 has three joints, and each of the joints is provided with an actuator to realize flexion and extension of the joint and rotation centered on the longitudinal direction of the arm unit.

The sensor 62 includes a force sensor configured to detect a force in a three-dimensional direction applied to the ultrasound probe 2; and a position sensor configured to detect the position of the robot arm 6. For example, the force sensor is a force senor of a strain gauge type, a piezoelectric type, or the like and is configured to detect a counterforce applied to the ultrasound probe 2 from the body surface of the subject. Further, the position sensor is a position sensor of a magnetic type, an angle type, an optical type, a rotation type, or the like, for example, and is configured to detect the position of the robot arm 6. In one example, the position sensor detects the position of the holding unit (the position of the ultrasound probe 2) of the robot arm 6, by detecting driven states of the joints. In other words, the position sensor detects the position of the ultrasound probe 2 within a three-dimensional movable range of the robot arm 6.

Further, for example, the position sensor detects the position of the holding unit (the position of the ultrasound probe 2) of the robot arm 6, by detecting the position of the position sensor with respect to a reference position. In one example, while the position sensor is provided in the holding unit of the robot arm 6, the position sensor detects the position of the ultrasound probe 2 within a space in which the ultrasound diagnosis process is performed, by detecting the position of the position sensor with respect to the reference position provided in the space in which the ultrasound diagnosis process is performed. The sensors described above are merely examples, and possible embodiments are not limited to these examples. In other words, as long as the sensors are able to obtain the position information of the robot arm 6, it is possible to use any type of sensors.

As illustrated in FIG. 2, the apparatus main body 5 includes the transmission and reception circuitry 51, B-mode processing circuitry 52, Doppler processing circuitry 53, storage 54, and processing circuitry 55. In the ultrasound diagnosis apparatus 1 illustrated in FIG. 2, processing functions are stored in the storage 54 in the form of computer-executable programs. The transmission and reception circuitry 51, the B-mode processing circuitry 52, the Doppler processing circuitry 53, and the processing circuitry 55 are processors configured to realize the functions corresponding to the programs by reading and executing the programs from the storage 54. In other words, each of the circuits that has read the corresponding one of the programs has the function corresponding to the read program.

The transmission and reception circuitry 51 includes a pulse generator, a transmission delay circuit, a pulser, and the like and is configured to supply the drive signal to the ultrasound probe 2. The pulse generator is configured to repeatedly generate a rate pulse used for forming a transmission ultrasound wave, at a predetermined rate frequency. Further, the transmission delay circuit is configured to apply a delay period that is required to converge the ultrasound wave generated from the ultrasound probe 2 into the form of a beam and to determine transmission directionality and that corresponds to each of the piezoelectric transducer elements, to each of the rate pulses generated by the pulse generator. Further, the pulser is configured to apply the drive signal (a drive pulse) to the ultrasound probe 2 with timing based on the rate pulses. In other words, by varying the delay periods applied to the rate pulses, the transmission delay circuit is able to arbitrarily adjust the transmission directions of the ultrasound waves transmitted from the surfaces of the piezoelectric transducer elements.

In this situation, the transmission and reception circuitry 51 has a function that is able to instantly change the transmission frequency, the transmission drive voltage, and the like, for the purpose of executing a predetermined scanning sequence on the basis of an instruction from the processing circuitry 55 (explained later). In particular, the function to change the transmission drive voltage is realized by using a linear-amplifier-type transmission circuitry of which the value can be instantly switched or by using a mechanism configured to electrically switch between a plurality of power source units.

Further, the transmission and reception circuitry 51 includes a pre-amplifier, an Analog/Digital (A/D) converter, a reception delay circuit, an adder, and the like and is configured to generate reflected-wave data by performing various types of processes on the reflected-wave signals received by the ultrasound probe 2. The pre-amplifier is configured to amplify the reflected-wave signal for each of the channels. The A/D converter is configured to perform an A/D conversion on the amplified reflected-wave signals. The reception delay circuit is configured to apply a delay period required to determine reception directionality thereto. The adder is configured to generate the reflected-wave data by performing an adding process on the reflected-wave signals processed by the reception delay circuit. As a result of the adding process performed by the adder, reflected components from the direction corresponding to the reception directionality of the reflected-wave signals are emphasized. A comprehensive beam for the ultrasound transmission and reception is formed according to the reception directionality and the transmission directionality.

The B-mode processing circuitry 52 is configured to generate data (B-mode data) in which signal intensities are expressed by degrees of brightness, by receiving the reflected-wave data from the transmission and reception circuitry 51 and performing a logarithmic amplification, an envelope detection process, and/or the like thereon.

The Doppler processing circuitry 53 is configured to generate data (Doppler data) obtained by extracting moving member information such as velocity, dispersion, power, and the like with respect to multiple points, by performing a frequency analysis to obtain velocity information from the reflected-wave data received from the transmission and reception circuitry 51 and extracting blood flows, tissues, contrast agent echo components based on the Doppler effect. Examples of the moving members in the present embodiment include fluids such as blood flowing through a blood vessel, lymph flowing through a lymphatic vessel, and the like.

In this situation, the B-mode processing circuitry 52 and the Doppler processing circuitry 53 are each capable of processing both two-dimensional reflected-wave data and three-dimensional reflected-wave data. In other words, the B-mode processing circuitry 52 is configured to generate two-dimensional B-mode data from two-dimensional reflected-wave data and to generate three-dimensional B-mode data from three-dimensional reflected-wave data. Further, the Doppler processing circuitry 53 is configured to generate two-dimensional Doppler data from two-dimensional reflected-wave data and to generate three-dimensional Doppler data from three-dimensional reflected-wave data. The three-dimensional B-mode data is data in which a brightness value is assigned in correspondence with the reflection intensity from a reflection source positioned at each of a plurality of points (sample points) set on the scanning lines in a three-dimensional scan range. Further, the three-dimensional Doppler data is data in which, to each of a plurality of points (sample points) set on the scanning lines in a three-dimensional scan range, a brightness value corresponding to a value of blood flow information (velocity, dispersion, power) is assigned.

The storage 54 is configured to store therein display-purpose image data generated by the processing circuitry 55. Further, the storage 54 is also capable of storing therein any of the data generated by the B-mode processing circuitry 52 and the Doppler processing circuitry 53. Further, the storage 54 stores therein control programs for performing ultrasound transmissions and receptions, image processing processes, and display processes as well as various types of data such as diagnosis information (e.g., subject's IDs, medical doctors' observations), diagnosis protocols, various types of body marks, and the like. Further, the storage 54 stores therein correspondence information in which a scan protocol is kept in correspondence with each diagnosed site. The correspondence information will be explained in detail later.

The processing circuitry 55 is configured to control overall processes performed by the ultrasound diagnosis apparatus 1. More specifically, the processing circuitry 55 performs various types of processes by reading and executing, from the storage 54, the programs corresponding to a controlling function 551, an image generating function 552, a robot controlling function 553, an analyzing function 554, and an output controlling function 555 illustrated in FIG. 2. In this situation, the processing circuitry 55 is an example of the processing circuitry.

For example, the processing circuitry 55 is configured to control processes performed by the transmission and reception circuitry 51, the B-mode processing circuitry 52, and the Doppler processing circuitry 53, on the basis of the various types of setting requests input by the operator via the input interface 4 and the various types of control programs and the various types of data read from the storage 54. Further, the processing circuitry 55 is configured to exercise control so that the monitor 3 displays display-purpose ultrasound image data stored in the storage 54. Further, the processing circuitry 55 is configured to exercise control so that the monitor 3 displays processing results. For example, by reading and executing a program corresponding to the controlling function 551, the processing circuitry 55 controls the entire apparatus so as to control the processes described above.

The image generating function 552 is configured to generate ultrasound image data from the data generated by the B-mode processing circuitry 52 and the Doppler processing circuitry 53. In other words, the image generating function 552 generates B-mode image data in which the intensities of the reflected waves are expressed with brightness levels, from the two-dimensional B-mode data generated by the B-mode processing circuitry 52. The B-mode image data is data rendering the shape of the tissue in the region on which the ultrasound scan was performed. Further, the image generating function 552 is configured to generate Doppler image data expressing the moving member information, from the two-dimensional Doppler data generated by the Doppler processing circuitry 53. The Doppler image data is velocity image data, dispersion image data, power image data, or image data combining any of these. The Doppler image data is data expressing fluid information related to the fluid flowing through the region on which the ultrasound scan was performed.

In this situation, generally speaking, the image generating function 552 converts (by performing a scan convert process) a scanning line signal sequence from an ultrasound scan into a scanning line signal sequence in a video format used by, for example, television and generates the display-purpose ultrasound image data. More specifically, the image generating function 552 generates the display-purpose ultrasound image data by performing a coordinate transformation process compliant with the ultrasound scan mode used by the ultrasound probe 2. Further, as various types of image processing processes besides the scan convert process, the image generating function 552 performs, for example, an image processing process (a smoothing process) to re-generate a brightness average value image, an image processing process (an edge enhancement process) that uses a differential filter inside an image, or the like, by using a plurality of image frames resulting from the scan convert process. Also, the image generating function 552 combines text information of various parameters, scale graduations, body marks, and the like, with the ultrasound image data.

In other words, the B-mode data and the Doppler data are each ultrasound image data before the scan convert process. The data generated by the image generating function 552 is the display-purpose ultrasound image data after the scan convert process. The B-mode data and the Doppler data may each be referred to as raw data.

Further, the image generating function 552 is configured to generate three-dimensional B-mode image data by performing a coordinate transformation process on the three-dimensional B-mode data generated by the B-mode processing circuitry 52. Further, the image generating function 552 is configured to generate three-dimensional Doppler image data by performing a coordinate transformation process on the three-dimensional Doppler data generated by the Doppler processing circuitry 53. The three-dimensional B-mode data and the three-dimensional Doppler data serve as volume data before the scan convert process. In other words, the image generating function 552 generates “three-dimensional B-mode image data and three-dimensional Doppler image data” as “volume data represented by three-dimensional ultrasound image data”.

Further, the image generating function 552 is capable of performing a rendering process on the volume data for the purpose of generating various types of two-dimensional image data used for displaying the volume data on the monitor 3.

An overall configuration of the ultrasound diagnosis apparatus 1 according to the first embodiment has thus been explained. The ultrasound diagnosis apparatus 1 according to the first embodiment configured as described above makes it possible to perform an ultrasound diagnosis process in a stable manner while having a scan performed by the robot. More specifically, when the robot arm 6 scans the subject, the ultrasound diagnosis apparatus 1 makes it possible to perform the ultrasound diagnosis process in a stable manner, by outputting various types of instructions for the subject.

During ultrasound diagnosis processes, instructions may be issued for the subjects in some situations regarding postures and respiration, depending on details of diagnoses and the status of the subjects. For example, during a diagnosis process on the abdomen, when the left lobe of the liver is observed by arranging the ultrasound probe 2 to approach from the costal arch, the technologist or the medical doctor instructs the subject to inhale so as to lower the diaphragm. As another example, when ultrasound image data includes an artifact caused by gas or a bone, the technologist or the medical doctor issues an instruction for the subject regarding the posture or respiration. Further, for example, during a diagnosis process performed on the locomotor system such as a tendon or a ligament, the technologist or the medical doctor instructs the subject to make a movement such as flexing and extending the joint and/or rotating the joint. Even when the robot arm 6 performs a scan on a subject, the ultrasound diagnosis apparatus 1 according to the first embodiment makes it possible to perform an ultrasound diagnosis in a stable manner, by precisely issuing an instruction for the subject in any of the abovementioned situations. In the following sections, details of processes performed by the ultrasound diagnosis apparatus 1 according to the first embodiment will be explained.

The robot controlling function 553 is configured to operate the robot arm 6 holding the ultrasound probe 2, by driving the mechanism unit 61 on the basis of information used for operating the robot arm 6 and the detection result obtained by the sensor 62 provided for the robot arm 6. More specifically, the robot controlling function 553 operates the robot arm 6 holding the ultrasound probe 2 so that the ultrasound probe 2 moves on the basis of a scan protocol indicating a scan procedure of the scan performed by the ultrasound probe 2. In this situation, the scan protocol is determined in advance in correspondence with each diagnosed site and stored in the storage 54. In other words, the robot controlling function 553 reads the scan protocol corresponding to the diagnosed site from the storage 54 and operates the robot arm 6 on the basis of the read scan protocol.

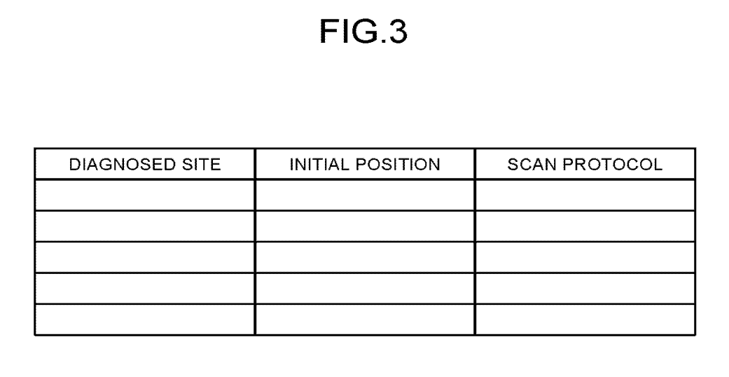

In this situation, the scan protocol is, for example, stored in the storage 54 as the correspondence information kept in correspondence with the relevant diagnosed site. FIG. 3 is a table illustrating an example of the correspondence information according to the first embodiment. As illustrated in FIG. 3, in the correspondence information, an initial position and a scan protocol are stored while being kept in correspondence with each of the diagnosed sites. In this situation, the “diagnosed site” denotes a site to be diagnosed in an ultrasound diagnosis process. Further, the “initial position” denotes a start position of the ultrasound probe 2 (i.e., the position, at the beginning, of the robot arm 6 holding the ultrasound probe 2) with respect to the scan performed by the robot arm 6. Further, the “scan protocol” denotes the procedure of each scan. In other words, the correspondence information illustrated in FIG. 3 is information in which, for each of the “diagnosed sites”, the start position of the ultrasound probe 2 and the procedure of moving the ultrasound probe 2 from the start position are kept in correspondence. For example, the robot controlling function 553 obtains information about the diagnosed site from a medical examination order or a diagnosis protocol input through the input interface 4 and moves the ultrasound probe 2 on the basis of the correspondence information illustrated in FIG. 3. In other words, the robot controlling function 553 moves the ultrasound probe 2 according to the scan protocol while using the “initial position” as the start position.

For example, the “initial position” is defined by the position of the ultrasound probe 2 with respect to the subject. In one example, a specific position at the diagnosed site (e.g., the lower end of the liver for an ultrasound examination performed on the abdomen) is defined as the “initial position”. The positional arrangement of the ultrasound probe 2 into the “initial position” may automatically be performed by analyzing an ultrasound image or may manually be performed by a technologist or a medical doctor. For example, when the positional arrangement is automatically performed, the technologist or the medical doctor, at first, arranges the ultrasound probe 2 held by the robot arm 6 to be in a position in the vicinity of the liver. The controlling function 551 and the image generating function 552 acquires an ultrasound image according to an instruction to start a scan.

In this situation, by operating the robot arm 6, the robot controlling function 553 moves the ultrasound probe 2 in an arbitrary direction from the initially arranged position. The controlling function 551 and the image generating function 552 acquire ultrasound images and transmit the acquired ultrasound images to the robot controlling function 553, even while the ultrasound probe 2 is being moved by the robot controlling function 553. The robot controlling function 553 brings the position in which the ultrasound probe 2 was initially arranged, into correspondence with the ultrasound image acquired at that time. Similarly, the robot controlling function 553 sequentially brings the positions into which the ultrasound probe 2 is moved, into correspondence with the ultrasound images acquired in those positions. Further, the robot controlling function 553 extracts the site from the sequentially-acquired ultrasound images and brings the extracted site into correspondence with the positions of the ultrasound probe 2. As a result, the robot controlling function 553 is able to establish an association about the positional relationship between the positions of the site of the subject and the space in which the ultrasound probe 2 is moved around. To extract the site from the ultrasound images, it is possible to use any of various types of existing algorithms.

Further, the robot controlling function 553 drives the robot arm 6 so that the ultrasound probe 2 is arranged in such a position where it is possible to scan the specific position set as the “initial position”. More specifically, by using the positional relationship of which the association was established as described above, the robot controlling function 553 determines a position corresponding to the specific position within the space in which the ultrasound probe 2 is moved around and arranges the ultrasound probe 2 so that the determined position is to be scanned. Alternatively, the robot controlling function 553 judges which position is being scanned at the current point in time by extracting the site from ultrasound images acquired while the robot arm 6 is further being operated and determines the direction of the specific position set as the “initial position” on the basis of the judgment result and anatomical position information of the site. After that, the robot controlling function 553 arranges the ultrasound probe 2 so that the specific position set as the “initial position” is to be scanned, by operating the robot arm 6 so as to move the ultrasound probe 2 in the determined direction. In this situation, the position of the ultrasound probe 2 within the space in which the ultrasound probe 2 is moved around is, as explained above, obtained by the sensor 62 provided for the robot arm 6 and forwarded, as a notification, to the robot controlling function 553.

In contrast, when the process of arranging the ultrasound probe 2 into the “initial position” is manually performed, the technologist or the medical doctor causes the ultrasound probe 2 held by the robot arm 6 to scan the vicinity of the liver and arranges the ultrasound probe 2 into the “initial position” while checking the position in the acquired ultrasound images. In this situation, the robot controlling function 553 establishes the association about the positional relationship between the position of the site in the body of the subject and the space in which the ultrasound probe 2 is moved around by detecting the site from the sequentially-acquired ultrasound images and bringing the detected site into correspondence with the position of the ultrasound probe 2 detected by the sensor 62.

As explained above, when the ultrasound probe 2 has been arranged in the “initial position”, the robot controlling function 553 moves the ultrasound probe 2 in the direction based on the scan protocol included in the correspondence information illustrated in FIG. 3. For example, the robot controlling function 553 operates the robot arm 6 so that the ultrasound probe 2 moves from the lower end of the liver toward the upper end side. In this situation, the robot controlling function 553 obtains, from the sensor 62, a counterforce applied to the ultrasound probe 2 from the body surface of the subject and further controls the robot arm 6 so that the obtained counterforce is substantially constant. In other words, the robot controlling function 553 operates the robot arm 6 while monitoring the counterforce applied to the ultrasound probe 2 from the body surface of the subject, so that the ultrasound probe 2 is not excessively pressed against the subject.

In the manner explained above, the ultrasound diagnosis apparatus 1 is configured so that the ultrasound probe 2 held by the robot arm 6 performs the scan corresponding to the diagnosed site on the subject. In this situation, the ultrasound diagnosis apparatus 1 according to the first embodiment outputs various instructions for the subject depending on details of diagnoses and the status of the subject. More specifically, the output controlling function 555 exercises control so that one or more instructions for the subject are output on the basis of the instruction information related to the ultrasound diagnosis.

For example, on the basis of the instruction information corresponding to the diagnosis protocol, the output controlling function 555 exercises control so that an instruction for the subject is output. During ultrasound diagnosis processes, the ultrasound probe 2 may perform a scan, in some situations, while the subject is changing his/her posture or holding his/her breath. In those situations, the technologist or the medical doctor would normally instruct the subject to change his/her posture so that ultrasound images are easily acquired or instruct the subject to hold his/her breath, while holding the ultrasound probe 2 and moving the ultrasound probe 2 along the body surface of the subject to acquire ultrasound images. In other words, the technologist or the medical doctor would instruct the subject to change the orientation of his/her body or the respiratory state, so that it is possible to acquire desired ultrasound images.

In the ultrasound diagnosis apparatus 1 according to the first embodiment, to issue the instructions as described above, the storage 54 stores therein a piece of instruction information for each diagnosis protocol. For example, for each diagnosis protocol, the storage 54 stores therein a piece of instruction information used for issuing an instruction about the posture or the respiratory state of the subject so as to be kept in correspondence therewith. In one example, the storage 54 may store therein the instruction information so as to be further kept in correspondence with the correspondence information illustrated in FIG. 3.

The output controlling function 555 is configured to exercise control so that, while the robot arm 6 is performing a scan, an instruction based on the instruction information is output for the subject. For example, the storage 54 stores therein the instruction information including the content of an instruction and the timing of the instruction so as to be kept in correspondence with the procedure for moving the ultrasound probe 2. While the robot arm 6 is being operated by the robot controlling function 553, the output controlling function 555 exercises control so that an instruction of which the content is stored is output with the instruction timing indicated in the instruction information. In one example, the output controlling function 555 exercises control so that, at the time when the moving of the ultrasound probe 2 by the robot controlling function 553 is stopped for a moment, an instruction to change the posture is output.

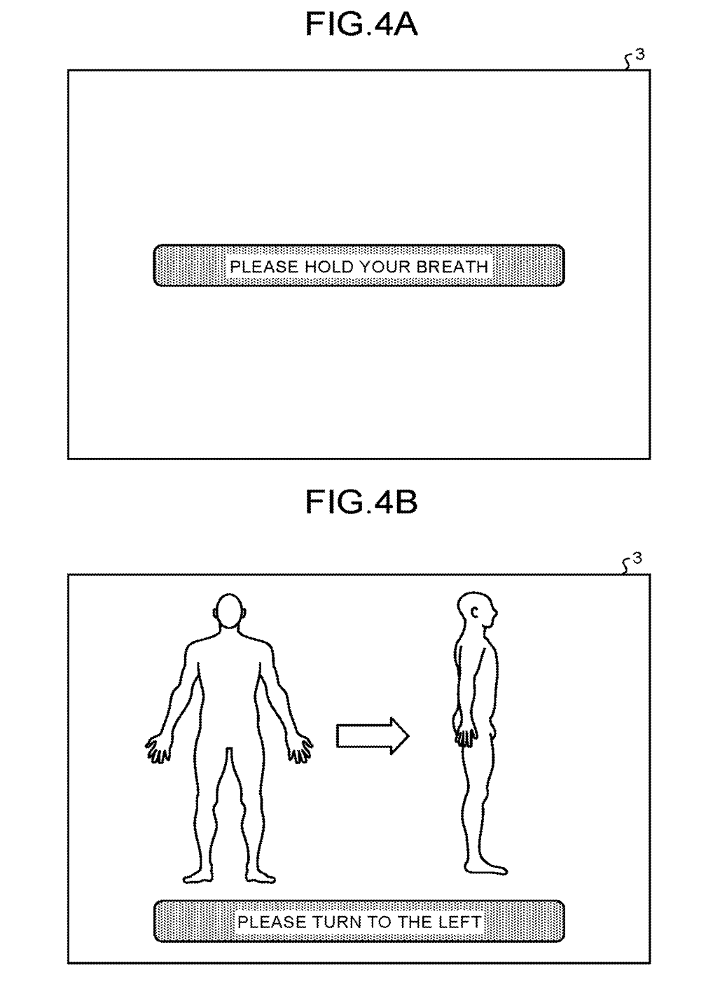

For example, the output controlling function 555 is configured to exercise control so that the instruction is output for the subject by using audio or display information. FIGS. 4A and 4B are drawings illustrating an example of the output information output by the output controlling function 555 according to the first embodiment. In this situation, FIGS. 4A and 4B illustrate an example in which the output controlling function 555 outputs an instruction by using the display information. For example, as illustrated in FIG. 4A, the output controlling function 555 is capable of causing the monitor 3 to display instruction information reading “Please hold your breath” with text. In this situation, the instruction information regarding respiration is not limited to the example presented above, but includes other various instructions such as “Please breathe in and hold your breath”, “Please breathe out and hold your breath”, and the like.

Further, for example, as illustrated in FIG. 4B, the output controlling function 555 is also capable of causing the monitor 3 to display instruction information including animation depicting changing the posture by turning to the left, in addition to an instruction using text that reads “Please turn to the left”. Further, the output controlling function 555 is also capable of exercising control so that an instruction using audio is output for the subject. In one example, the output controlling function 555 exercises control so that an audio message “Please turn to the left” is output from a speaker provided for the monitor 3.

As explained above, the ultrasound diagnosis apparatus 1 according to the first embodiment is configured to issue the instructions for the subject by using the text information, the animation, the audio, and the like. The output controlling function 555 is also capable of outputting any of the text information, the animation, the audio, and the like, in combination, as appropriate. In other words, the output controlling function 555 is capable of outputting an instruction by using one selected from among the text information, the animation, and the audio, and is also capable of outputting an instruction by combining together any of the plurality of methods (e.g., the animation and the text information), as illustrated in FIG. 4B.

In the explanation above, the example is explained in which the instruction is output with the timing kept in correspondence with the diagnosis protocol. However, the output controlling function 555 is also capable of outputting instruction information in accordance with the position of the robot arm 6 with respect to the subject. For example, on the basis of the position of the ultrasound probe 2 with respect to the subject detected by the sensor 62 provided for the robot arm 6, the output controlling function 555 may output an instruction for the subject to change his/her posture. In that situation, for example, the storage 54 stores therein pieces of instruction information so as to be kept in correspondence with positions of the ultrasound probe 2 with respect to the subject. The output controlling function 555 compares information about the position provided by the sensor 62 while the ultrasound probe 2 is performing a scan with the information stored in the storage 54, and when the position of the ultrasound probe 2 with respect to the subject corresponds to one of the stored positions, the output controlling function 555 outputs the corresponding instruction for the subject.

Further, the output controlling function 555 is also capable of analyzing an acquired ultrasound image and outputting instruction information on the basis of the result of the analysis. More specifically, the analyzing function 554 is configured to make an analysis as to whether or not an instruction is to be output for the subject, by comparing one or more ultrasound images acquired from the subject by the ultrasound probe 2 held by the robot arm 6 with ultrasound images stored in advance in correspondence with positions of the robot arm 6 with respect to the subject.

For example, the storage 54 stores therein, in advance, ultrasound images acquired in such positions where gas or a bone is included therein. By comparing sequentially-acquired ultrasound images with the ultrasound images stored in the storage 54, the analyzing function 554 judges whether or not any of the acquired ultrasound images include gas or a bone. For example, the analyzing function 554 judges whether or not any of the acquired ultrasound images include gas or a bone, by performing a pattern matching process on the pixel values between the acquired ultrasound images and the ultrasound images stored in advance.

When it is determined that one or more of the acquired ultrasound images include gas or a bone, the output controlling function 555 outputs an instruction for the subject. For example, when one or more of the acquired ultrasound images include gas, the output controlling function 555 outputs an instruction regarding respiration. In another example, when one or more of the sequentially-acquired ultrasound images include a bone, the output controlling function 555 outputs an instruction regarding the posture.

In this situation, the result of the analysis performed by the analyzing function 554 may be used not only for judging whether or not an instruction is to be output for the subject, but also for controlling the position of the ultrasound probe 2. In other words, when it is determined that one or more of the acquired ultrasound images include gas or a bone, the robot controlling function 553 operates the robot arm 6, so as to eliminate the gas or the bone from the ultrasound images. For example, the robot controlling function 553 moves the ultrasound probe 2, so that the gas and/or the bone will not be included in the ultrasound images, in accordance with the position of the gas and/or the bone rendered in the ultrasound images.

The instructions issued for the subject by the ultrasound diagnosis apparatus 1 have thus been explained. In this situation, the ultrasound diagnosis apparatus 1 is also capable of stopping the operation of the robot arm 6, in response to an input from the subject. For example, when the subject utters a sound indicating an abnormality into the microphone included in the input interface 4, the robot controlling function 553 stops the operation of the robot arm 6. Further, for example, when the subject presses a button or the like included in the input interface 4, the robot controlling function 553 stops the operation of the robot arm 6.

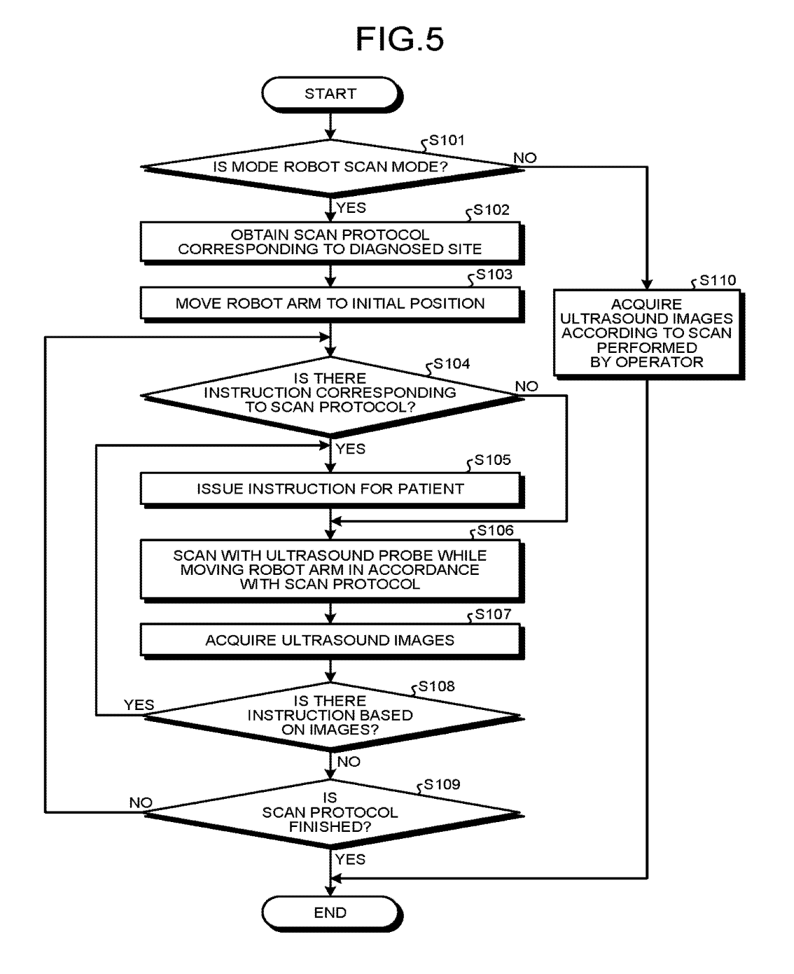

Next, a process performed by the ultrasound diagnosis apparatus 1 according to the first embodiment will be explained, with reference to FIG. 5. FIG. 5 is a flowchart for explaining a procedure in a process performed by the ultrasound diagnosis apparatus 1 according to the first embodiment. Step S101, step S107, step S109, and step S110 illustrated in FIG. 5 are steps executed as a result of the processing circuitry 55 reading the program corresponding to the controlling function 551 from the storage 54. Step S102, step S103, and step S106 are steps executed as a result of the processing circuitry 55 reading the program corresponding to the robot controlling function 553 from the storage 54. Step S104 and step S105 are steps executed as a result of the processing circuitry 55 reading the program corresponding to the output controlling function 555 from the storage 54. Step S108 is a step executed as a result of the processing circuitry 55 reading the program corresponding to the analyzing function 554 from the storage 54.

At step S101, the processing circuitry 55 judges whether or not the current mode is a robot scan mode. When the current mode is not the robot scan mode (step S101: No), the processing circuitry 55 acquires ultrasound images according to a scan performed by the operator (step S110). On the contrary, when the current mode is the robot scan mode (step S101: Yes), the processing circuitry 55 obtains a scan protocol corresponding to the diagnosed site (step S102) and moves the robot arm 6 to the initial position (step S103).

Subsequently, at step S104, the processing circuitry 55 judges whether or not there is an instruction corresponding to the scan protocol. When there is an instruction corresponding to the scan protocol (step S104: Yes), the processing circuitry 55 outputs the instruction for the subject (step S105) and scans the subject with the ultrasound probe 2 while moving the robot arm 6 according to the scan protocol (step S106). On the contrary, when there is no instruction corresponding to the scan protocol (step S104: No), the processing circuitry 55 scans the subject with the ultrasound probe 2, while moving the robot arm 6 according to the scan protocol (step S106).

Further, the processing circuitry 55 acquires one or more ultrasound images (step S107). At step S108, the processing circuitry 55 judges whether or not there is an instruction based on the images. When there is an instruction based on the images (step S108: Yes), the processing circuitry 55 returns to step S105 and outputs the instruction for the subject. On the contrary, when there is no instruction based on the images (step S108: No), the processing circuitry 55 judges whether or not the scan protocol is finished at step S109.

When it is determined that the scan protocol is finished (step S109: Yes), the processing circuitry 55 ends the process. On the contrary, when it is determined that the scan protocol is not finished (step S109: No), the processing circuitry 55 returns to step S104 and judges whether or not there is an instruction corresponding to the scan protocol.

As explained above, according to the first embodiment, the ultrasound probe 2 is configured to transmit and receive the ultrasound wave. The robot arm 6 is configured to hold the ultrasound probe 2 and to move the ultrasound probe 2 along the body surface of the subject. The robot controlling function 553 is configured to control the moving of the ultrasound probe 2 performed by the robot arm 6. The output controlling function 555 is configured to exercise control so that the one or more instructions are output for the subject on the basis of the instruction information. Consequently, the ultrasound diagnosis apparatus 1 according to the first embodiment is able to issue the instructions for the subject. It is therefore possible to perform the ultrasound diagnosis process in a stable manner while having the scan performed by the robot.

Further, in the first embodiment, the output controlling function 555 is configured to output the one or more instruction for the subject, on the basis of the instruction information corresponding to the diagnosis protocol. Consequently, the ultrasound diagnosis apparatus 1 according to the first embodiment is able to issue instructions for the subject even during an ultrasound diagnosis process that requires the subject to make a movement. It is therefore possible to perform the ultrasound diagnosis process in a more stable manner.

Further, in the first embodiment, the output controlling function 555 is configured to output the one or more instructions for the subject on the basis of the instruction information corresponding to the position of the robot arm 6 with respect to the subject. Consequently, the ultrasound diagnosis apparatus 1 according to the first embodiment is able to issue the instructions corresponding to the positional state of the subject and the robot arm 6 during the scan performed by the robot arm 6. It is therefore possible to perform the ultrasound diagnosis process in a more stable manner.

Further, in the first embodiment, the analyzing function 554 is configured to make the analysis as to whether or not an instruction is to be output for the subject, by comparing the ultrasound images acquired from the subject by the ultrasound probe 2 held by the robot arm 6, with the ultrasound images stored, in advance, in correspondence with the positions of the robot arm 6 with respect to the subject. The output controlling function 555 is configured to output the one or more instructions for the subject on the basis of the result of the analysis made by the analyzing function 554. Consequently, the ultrasound diagnosis apparatus 1 according to the first embodiment is able to output the instructions based on the ultrasound images. It is therefore possible to perform the ultrasound diagnosis process in a more stable manner.

Further, in the first embodiment, the output controlling function 555 is configured to output the one or more instructions for the subject by using the audio or the display information. Consequently the ultrasound diagnosis apparatus 1 according to the first embodiment makes it possible to precisely issue the instructions in various situations.

Further, in the first embodiment, the input interface 4 is configured to receive the input from the subject. When the input interface 4 receives the input from the subject, the robot controlling function 553 is configured to stop the robot arm 6 from moving. Consequently, the ultrasound diagnosis apparatus 1 according to the first embodiment makes it possible to have the scan performed by the robot more safely.

### Second Embodiment

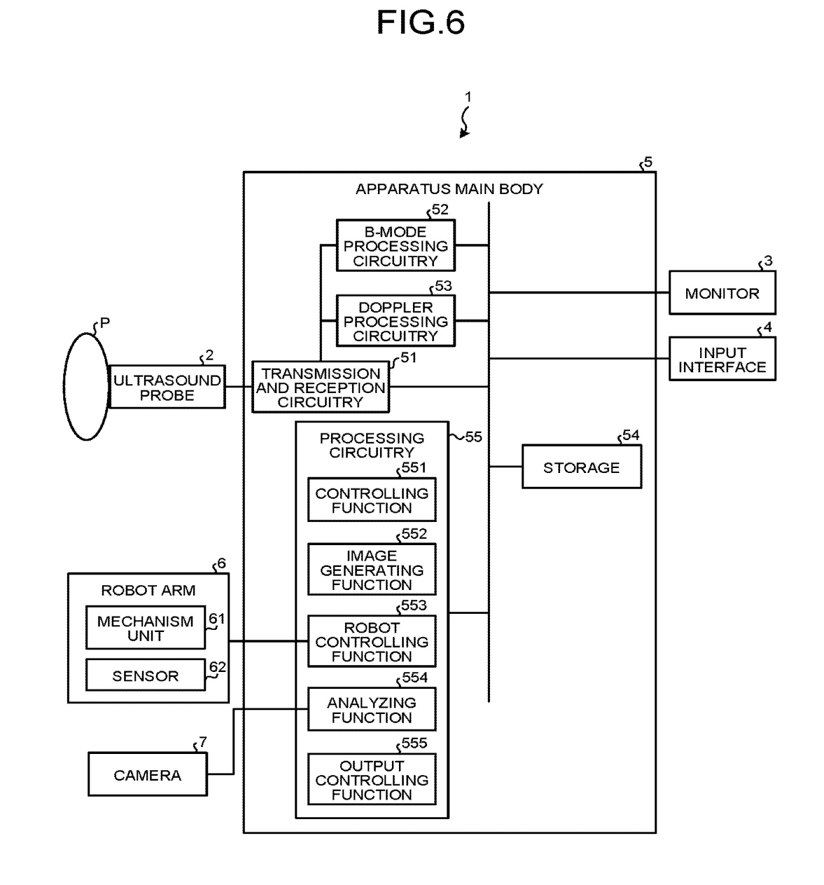

In a second embodiment, an example will be explained in which it is judged whether or not an instruction is to be output for the subject, by using a picture obtained by imaging the state of the subject and the robot arm 6 with a camera. FIG. 6 is a block diagram illustrating an exemplary configuration of the ultrasound diagnosis apparatus 1 according to the second embodiment. The ultrasound diagnosis apparatus 1 according to the second embodiment is different from that in the first embodiment for having a camera 7 connected thereto, for the process performed by the analyzing function 554, and for the information stored by the storage 54. In the following sections, the second embodiment will be explained while a focus is placed on these differences.

In the ultrasound diagnosis apparatus 1 according to the second embodiment, the camera 7 is configured to acquire a picture exhibiting a positional relationship between the subject and the robot arm 6 (the ultrasound probe 2) and to transmit the acquired picture to the analyzing function 554. For example, the camera 7 is disposed in a room in which the ultrasound diagnosis process is performed and is connected to the ultrasound diagnosis apparatus 1. Further, the camera 7 acquires the picture of the scan performed on the subject by the robot arm 6 and transmits the acquired picture to the ultrasound diagnosis apparatus 1.

In correspondence with each of multiple positions of the ultrasound probe 2 with respect to the subject, the storage 54 is configured to store therein an ultrasound image acquired in the position. In other words, the storage 54 is configured to store therein information in which ultrasound images suitable for observation are kept in correspondence with the positions in which the ultrasound probe 2 was located with respect to the subject when those ultrasound images were acquired. In this situation, this type of information may be stored for each subject or for each of various common physiques. For example, the storage 54 stores therein reference information in which, for each subject, ultrasound images suitable for observation are kept in correspondence with the positions in which the ultrasound probe 2 was located with respect to the subject when those ultrasound images were acquired.

In this situation, it is possible to update the reference information stored in the storage 54 as appropriate by using a learning function. For example, the analyzing function 554 brings pictures taken during an ultrasound diagnosis process, as well as positions of the robot arm 6 during the ultrasound diagnosis process and the acquired ultrasound images into correspondence with one another, in a time series. After that, the analyzing function 554 stores, into the storage 54, ultrasound images used for diagnosis or analysis purposes so as to be kept in correspondence with the positions in which the ultrasound probe 2 was located with respect to the subject when those ultrasound images were taken. In this manner, every time an ultrasound diagnosis process is performed, the analyzing function 554 updates the reference information in which the ultrasound images suitable for observation are kept in correspondence with the positions in which the ultrasound probe 2 was located with respect to the subject when those ultrasound images were taken. Details of the process of updating the reference image will be explained later.

The analyzing function 554 is configured to judge whether or not an instruction is to be output for the subject by obtaining, from the camera 7, a picture of the current point in time while a scan is being performed on the subject and comparing the obtained picture with the reference information stored in the storage 54. More specifically, the analyzing function 554 reads a piece of reference information that has an ultrasound image of the diagnosed site of the current point in time kept in correspondence and further compares the position of the ultrasound probe 2 with respect to the subject kept in correspondence in the read piece of reference information with the position of the ultrasound probe 2 with respect to the subject at the current point in time. Further, when the difference between the read position and the position in the current point in time exceeds a threshold value, the analyzing function 554 determines that an instruction is to be output for the subject. Subsequently, the analyzing function 554 notifies the output controlling function 555 of information about the difference between the read position and the position in the current point in time.

On the basis of the result of the judgment made by the analyzing function 554, the output controlling function 555 is configured to exercise control so as to output an instruction for the subject. For example, the output controlling function 555 instructs the subject to change his/her posture so that the difference in the positions analyzed by the analyzing function 554 becomes equal to or smaller than the predetermined threshold value. In one example, the output controlling function 555 instructs the subject to move his/her body in such a direction that solves the positional difference, on the basis of the information about the difference indicated in the notification from the analyzing function 554.

As explained above, the ultrasound diagnosis apparatus 1 according to the second embodiment is configured to determine the position of the robot arm 6 with respect to the subject, on the basis of the pictures taken by the camera 7 and to output the instruction for the subject on the basis of the determined position and the reference information. In this situation, as mentioned above, it is possible to update the reference information as appropriate by using the learning function. For example, when updating the reference information for each subject, the ultrasound diagnosis apparatus 1, at first, causes the robot arm 6 to perform a scan on the basis of the reference information corresponding to the subject that has already been stored in the storage 54.

In this situation, the analyzing function 554 extracts a diagnosed site with respect to each of the ultrasound images acquired during the scan and compares each image with the ultrasound images kept in correspondence within the reference information. After that, the analyzing function 554 stores, into the storage 54, an ultrasound image rendering the diagnosed site more clearly than the already-stored ultrasound images and the picture corresponding to the time when the ultrasound image was acquired (the position of the ultrasound probe 2 with respect to the subject), as a new piece of reference information. In this situation, it is acceptable to judge whether or not the diagnosed site is rendered more clearly, on the basis of an occupancy ratio of the diagnosed site in the image (the size of the diagnosed site within the image) or the level of image contrast, for example.

Further, for example, when updating the reference information for each of the various physiques, the ultrasound diagnosis apparatus 1 causes the robot arm 6 to perform a scan on the basis of the reference information of the corresponding physique that has already been stored in the storage 54. After that, by performing the same process as described above, the ultrasound diagnosis apparatus 1 updates the reference information of the corresponding physique that has already been stored.

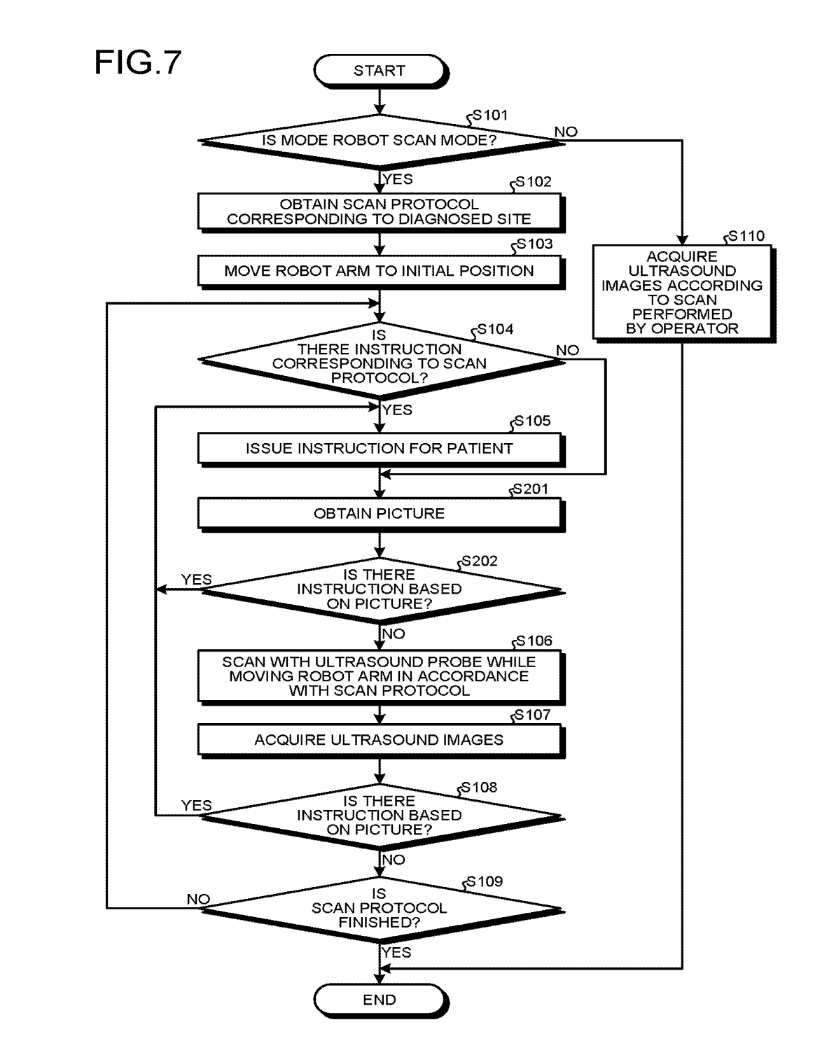

Next, a process performed by the ultrasound diagnosis apparatus 1 according to the second embodiment will be explained with reference to FIG. 7. FIG. 7 is a flowchart for explaining a procedure in the process performed by the ultrasound diagnosis apparatus 1 according to the second embodiment. The flowchart illustrated in FIG. 2 has steps S201 and S202 added to the flowchart illustrated in FIG. 5. In the following sections, the procedure will be explained while a focus is placed on these steps. Step S201 and step S202 illustrated in FIG. 7 are steps executed as a result of the processing circuitry 55 reading the program corresponding to the analyzing function 554 from the storage 54.

At step S101, when the current mode is not the robot scan mode (step S101: No), the processing circuitry 55 acquires ultrasound image images according to a scan performed by the operator (step S110). On the contrary, when the current mode is the robot scan mode (step S101: Yes), the processing circuitry 55 obtains a scan protocol corresponding to the diagnosed site (step S102) and moves the robot arm 6 to the initial position (step S103).

Subsequently, at step S104, when there is an instruction corresponding to the scan protocol (step S104: Yes), the processing circuitry 55 outputs an instruction for the subject (step S105). In this situation, in the ultrasound diagnosis apparatus 1 according to the second embodiment, the processing circuitry 55 obtains a picture from the camera 7 (step S201) and judges whether or not there is an instruction based on the picture (step S202). When it is determined that there is an instruction based on the picture (step S202: Yes), the processing circuitry 55 returns to step S105 and outputs the instruction for the subject.

On the contrary, when it is determined that there is no instruction based on the picture (step S202: No), the processing circuitry 55 scans the subject with the ultrasound probe 2 while moving the robot arm 6 according to the scan protocol (step S106). Further, the processing circuitry 55 acquires one or more ultrasound images (step S107) and judges whether or not there is an instruction based on the images (step S108). When there is an instruction based on the images (step S108: Yes), the processing circuitry 55 returns to step S105 and outputs the instruction for the subject. On the contrary, when there is no instruction based on the images (step S108: No), the processing circuitry 55 judges whether or not the scan protocol is finished at step S109.

When it is determined that the scan protocol is finished (step S109: Yes), the processing circuitry 55 ends the process. On the contrary, when it is determined that the scan protocol is not finished (step S109: No), the processing circuitry 55 returns to step S104 and judges whether or not there is an instruction corresponding to the scan protocol. At step S104, when there is no instruction corresponding to the scan protocol (step S104: No), the processing circuitry 55 proceeds to step S201 and obtains a picture.

As explained above, according to the second embodiment, the analyzing function 554 is configured to make the analysis as to whether or not an instruction is to be output for the subject, by comparing the picture taken of the subject and the robot arm 6 with the reference image being stored in advance and indicating the positional relationship between the subject and the robot arm 6. The output controlling function 555 is configured to output the instruction for the subject on the basis of the result of the analysis made by the analyzing function 554. Consequently, the ultrasound diagnosis apparatus 1 according to the second embodiment is able to issue the instructions for the subject by using the more accurate position information. It is therefore possible to perform the ultrasound diagnosis process in a more stable manner.

### Third Embodiment

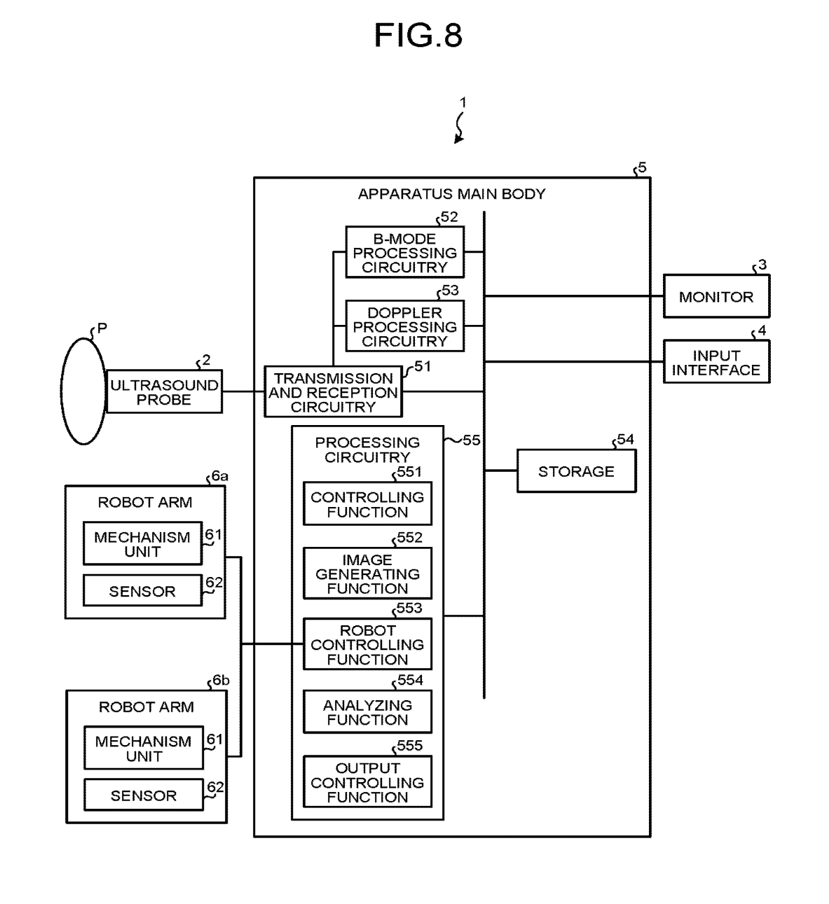

In a third embodiment, an example will be explained in which a plurality of robot arms are provided. FIG. 8 is a block diagram illustrating an exemplary configuration of the ultrasound diagnosis apparatus 1 according to the third embodiment. The ultrasound diagnosis apparatus 1 according to the third embodiment is different from that in the first embodiment for having the plurality of robot arms. In the following sections, the third embodiment will be explained while a focus is placed on the difference.

As illustrated in FIG. 8, the ultrasound diagnosis apparatus 1 according to the third embodiment includes a robot arm 6a and another robot arm 6b. In this situation, the robot arms 6a and 6b may be robot arms configured to perform mutually the same operation or may be robot arms configured to perform mutually-different operations. In other words, the robot arms 6a and 6b may both be the same as the robot arm 6 described above. In that situation, for example, the robot arm 6a and the robot arm 6b each hold an ultrasound probe 2 of mutually the same type. Alternatively, for example, the robot arm 6a and the robot arm 6b may hold ultrasound probes 2 of mutually-different types. In another example, the robot arm 6a and the robot arm 6b may hold one ultrasound probe in collaboration with each other.

Further, one of the robot arms 6a and 6b may be the same as the robot arm 6 described above, while the other may be a robot arm of a different type from the robot arm 6. In that situation, for example, it is acceptable to adopt a support arm as the robot arm of the different type. In this situation, for example, the support arm provides support for diagnosing blood flows. In one example, one of the robot arms 6a and 6b functions as a support arm that presses a vein during a blood flow diagnosis process performed by implementing a vein pressure method.

The robot controlling function 553 is configured to control operations performed on the subject by the support arm. For example, the robot controlling function 553 controls the process of pressing of the vein performed by the support arm. In this situation, the output controlling function 555 is also capable of outputting an instruction for the subject, on the basis of a relative position between the support arm and the subject. For example, the output controlling function 555 is capable of instructing the subject to “extend his/her knee”.

As explained above, according to the third embodiment, the robot controlling function 553 is further configured to control the operations performed on the subject by the support arm. The output controlling function 555 is configured to exercise control so that one or more instructions are output for the subject on the basis of instruction information related to manipulations using the support arm. Consequently, the ultrasound diagnosis apparatus 1 according to the third embodiment is able to control the plurality of robot arms. It is therefore possible to apply the scans performed by the robot, to various types of manipulations.

### Fourth Embodiment

The first to the third embodiments have thus been explained. Further, it is possible also to carry out the present disclosure in various different forms other than those described in the first to the third embodiments.

In the embodiments above, the example is explained in which the ultrasound probe 2 is connected to the apparatus main body 5 via the cable; however, possible embodiments are not limited to this example. For instance, the transmission and the reception of the ultrasound waves by the ultrasound probe may be controlled wirelessly. In that situation, for example, the probe main body of the ultrasound probe has transmission and reception circuitry built therein so that the transmission and the reception of the ultrasound waves by the ultrasound probe are controlled wirelessly by another apparatus. The ultrasound diagnosis apparatus according to the present embodiments may be configured so as to include only such a wireless ultrasound probe.

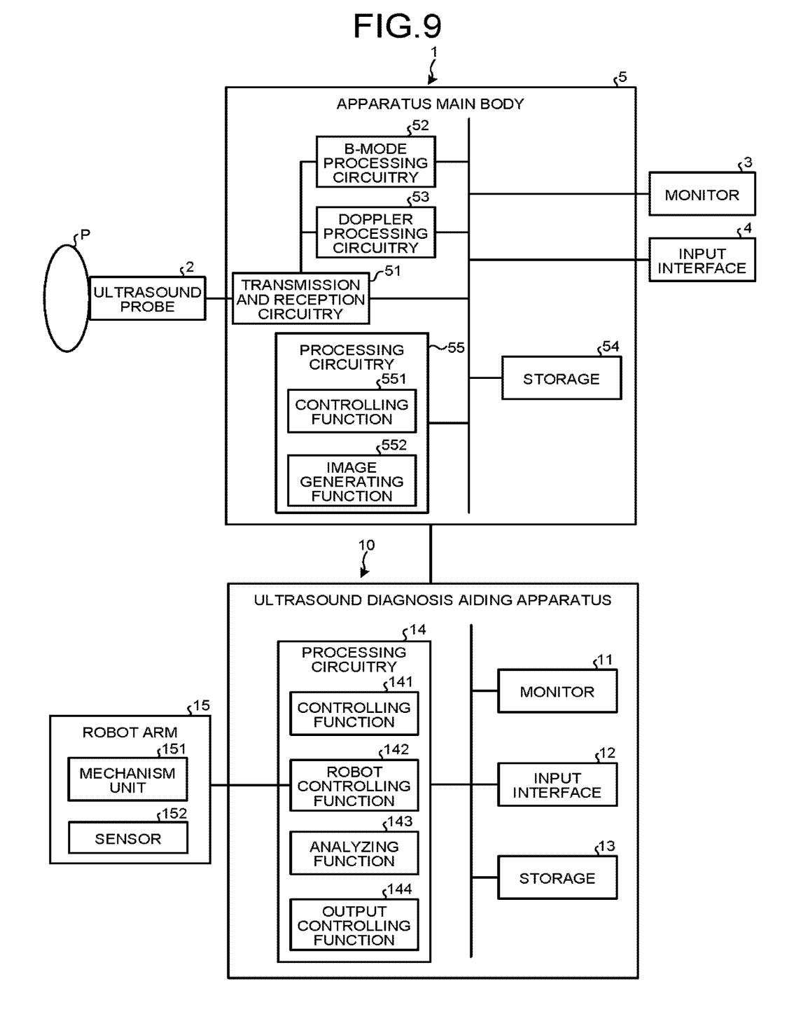

In the embodiments described above, the example is explained in which the ultrasound diagnosis apparatus 1 performs the various types of processes; however, possible embodiments are not limited to this example. For instance, an ultrasound diagnosis aiding apparatus may perform the various types of processes. FIG. 9 is a diagram illustrating an exemplary configuration of an ultrasound diagnosis aiding apparatus 10 according to a fourth embodiment. As illustrated in FIG. 9, the ultrasound diagnosis aiding apparatus 10 according to the fourth embodiment includes a monitor 11, an input interface 12, storage 13, processing circuitry 14, and a robot arm 15 and is connected to the ultrasound diagnosis apparatus 1.

The monitor 11 is configured to display a Graphical User Interface (GUI) used by an operator of the ultrasound diagnosis aiding apparatus 10 to input various types of setting requests through the input interface 12 and to display processing results obtained by the processing circuitry 14 and the like. Further, the monitor 11 is configured to output the instruction information for the subject on the basis of control exercised by the processing circuitry 14. For example, the monitor 11 displays the instruction information realized with text, animation, or the like as described above, for the subject. Further, for example, the monitor 11 is configured to output the instruction information realized with audio from a speaker built therein, as described above.

The input interface 12 is realized by using a mouse, a keyboard, a button, a panel switch, a microphone, and/or the like. The input interface 12 is configured to receive the various types of setting requests from the operator of the ultrasound diagnosis aiding apparatus 10 and to transfer the received various types of setting requests to the processing circuitry 14. Further, the input interface 12 is configured to receive a request from the subject and to transfer the received request to the processing circuitry 14. The storage 13 is configured to store therein various types of information similar to the information stored in the storage 54 described above.

The processing circuitry 14 is configured to control overall processes performed by the ultrasound diagnosis aiding apparatus 10. More specifically, the processing circuitry 14 performs various types of processes by reading and executing, from the storage 13, programs corresponding to a controlling function 141, a robot controlling function 142, an analyzing function 143, and an output controlling function 144 illustrated in FIG. 9. In other words, the processing circuitry 14 is a processor configured to realize the functions corresponding to the programs by reading and executing the programs from the storage 13. That is to say, the processing circuitry 14 that has read the programs has the functions corresponding to the read programs. In this situation, the robot controlling function 142 is an example of the robot controlling unit set forth in the claims. The analyzing function 143 is an example of the analyzing unit set forth in the claims. The output controlling function 144 is an example of the output controlling unit set forth in the claims.

The controlling function 141 is configured to control various types of processes performed by the ultrasound diagnosis aiding apparatus 10. Further, the controlling function 141 is configured to obtain ultrasound images from the ultrasound diagnosis apparatus 1. The robot controlling function 142, the analyzing function 143, and the output controlling function 144 are configured to perform the same processes as those performed by the robot controlling function 553, the analyzing function 554, and the output controlling function 555 described above. The robot arm 15 includes a mechanism unit 151 and a sensor 152. Further, the robot arm 15 is configured to hold the ultrasound probe 2 connected to the ultrasound diagnosis apparatus 1 and is controlled in the same manner as the robot arm 6 and the like are as explained above.

The term “processor” used in the explanation above denotes, for example, a Central Processing Unit (CPU), a Graphics Processing Unit (GPU), or a circuit such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). Each of the processors realizes the functions thereof by reading and executing a corresponding one of the programs stored in storage. In this situation, instead of saving the programs in the storage, it is also acceptable to directly incorporate the programs in the circuits of the processors. In that situation, each of the processors realizes the functions thereof by reading and executing the corresponding one of the programs incorporated in the circuit thereof. Further, the processors in the present embodiments do not each necessarily have to be structured as a single circuit. It is also acceptable to structure one processor by combining together a plurality of independent circuits so as to realize the functions thereof.

The constituent elements of the apparatuses and the devices illustrated in the drawings used in the explanations of the embodiments above are based on functional concepts. Thus, it is not necessary to physically configure the constituent elements as indicated in the drawings. In other words, the specific modes of distribution and integration of the apparatuses and the devices are not limited to those illustrated in the drawings. It is acceptable to functionally or physically distribute or integrate all or a part of the apparatuses and the devices in any arbitrary units, depending on various loads and the status of use. Further, all or an arbitrary part of the processing functions performed by the apparatuses and the devices may be realized by a CPU and a program that is analyzed and executed by the CPU or may be realized as hardware using wired logic.

Further, the processing methods explained in the above embodiments may be realized by causing a computer such as a personal computer or a workstation to execute a processing program prepared in advance. The processing program may be distributed via a network such as the Internet. Further, the processing program may be recorded on a computer-readable non-transitory recording medium such as a hard disk, a flexible disk (FD), Compact Disk Read-Only Memory (CD-ROM), a Magneto-Optical (MO) disk, a Digital Versatile Disk (DVD), or a flash memory such as a Universal Serial Bus (USB) memory, a Secure Digital (SD) card memory or the like, so as to be executed as being read from the non-transitory recording medium by a computer.

As explained above, according to at least one aspect of the embodiments, it is possible to perform the ultrasound diagnosis process in a stable manner while having the scan performed by the robot.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus comprising:
an ultrasound probe configured to transmit and receive an ultrasound wave;
a robot arm configured to hold the ultrasound probe and to move the ultrasound probe along a body surface of a subject; and
processing circuitry configured to control the moving of the ultrasound probe performed by the robot arm and to exercise control so that an instruction for the subject is output on a basis of instruction information related to an ultrasound diagnosis.

2. The ultrasound diagnosis apparatus according to claim 1, wherein the processing circuitry is configured to output the instruction for the subject on the basis of the instruction information corresponding to a diagnosis protocol.

3. The ultrasound diagnosis apparatus according to claim 1, wherein the processing circuitry is configured to output the instruction for the subject on the basis of the instruction information corresponding to a position of the robot arm with respect to the subject.

4. The ultrasound diagnosis apparatus according to claim 3, wherein the processing circuitry is configured to
make an analysis as to whether or not the instruction is to be output for the subject, by comparing a picture taken of the subject and the robot arm with a reference image being stored in advance and indicating a positional relationship between the subject and the robot arm, and
output the instruction for the subject on a basis of a result of the analysis.

5. The ultrasound diagnosis apparatus according to claim 1, wherein the processing circuitry is configured to
make an analysis as to whether or not the instruction is to be output for the subject, by comparing an ultrasound image acquired from the subject by the ultrasound probe held by the robot arm, with ultrasound images stored in advance while being kept in correspondence with positions of the robot arm with respect to the subject, and
output the instruction for the subject on a basis of a result of the analysis.

6. The ultrasound diagnosis apparatus according to claim 1, wherein the processing circuitry is configured to output the instruction for the subject by using audio or display information.

7. The ultrasound diagnosis apparatus according to claim 1, further comprising: a support arm configured to perform an operation on the subject, wherein
the processing circuitry is further configured to control the operation performed on the subject by the support arm.

8. The ultrasound diagnosis apparatus according to claim 1, further comprising: an input interface configured to receive an input from the subject, wherein
when the input interface is configured to receive the input from the subject, the processing circuitry is configured to stop the robot arm from moving.

9. An ultrasound diagnosis aiding apparatus comprising:
a robot arm configured to hold an ultrasound probe that transmits and receives an ultrasound wave and to move the ultrasound probe along a body surface of a subject; and
processing circuitry configured to control the moving of the ultrasound probe performed by the robot arm and to output an instruction for the subject on a basis of instruction information related to an ultrasound diagnosis.
